(19) United States
(12) Patent Application Publication
(10) Pub. No.: US 2010/0212548 A1
(43) Pub. Date: Aug. 26, 2010

(54) Immersion Liquid And Method For Preparing An Organic Compound For An Immersion Liquid

(76) Inventor: Hans-Joachim Weippert, Aalen (DE)

Correspondence Address: FITCH EVEN TABIN & FLANNERY, 120 SOUTH LASALLE STREET, SUITE 1600, CHICAGO, IL, 60603-3406, US

(21) Appl. No.: 12/712,000

(22) Filed: Feb. 24, 2010

(30) Foreign Application Priority Data
Feb. 25, 2009 (DE) 10 2009 010 504.2

(51) Int. Cl. C09D 5/00; G02B 21/33; C07C 333/00

(52) U.S. Cl. 106/287.3; 558/237

(57) ABSTRACT

The invention relates to an immersion liquid including at least one organic compound having at least one structural element of the general formula I

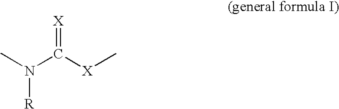

wherein X denotes independently of each other S or O and R denotes hydrogen or hydrocarbon. The invention further relates to a method for preparing an organic compound for an immersion liquid as well as a use of the immersion liquid and the organic compound as an immersion oil for a microscope.

### TECHNICAL FIELD

The invention relates to an immersion liquid, a method for preparing an organic compound for an immersion liquid as well as a use of the immersion liquid and of the organic compound.

### PRIOR ART

Light collecting efficiency and resolution in the microscopy are directly dependent on the numerical aperture (NA) of the employed objective. A major problem for example of the single molecule microscopy in living cells is in the autofluorescence of diverse cell components such as for example proteins, cell metabolites and the like. For examining such cells, often, the so-called total internal reflection microscopy (TIRF) is used, which is a wide-field technique. In the total internal reflection fluorescence microscopy, the NA including the refractive indices of immersion oil and cover glass moreover also determines the penetration depth of the excitation light if the illumination is effected through the objective. A particularly large NA then allows illuminating extremely thin layers.

In order to further increase the NAs of about 1.49 achievable with conventional immersion oil heretofore, for the development of immersion objectives with high NA for the near-field microscopy, compounds or mixtures of compounds with larger refractive index are necessary. In addition, the compounds are to satisfy various further requirements. Thus, besides the refractive index as high as possible, above all, a low intrinsic fluorescence, a low volatility as well as a low toxicity are desirable.

From the prior art, various highly refractive compounds and immersion liquids are known. For example, US 2008/0135808 A1 discloses an immersion oil, which is composed of a mixture of diiodomethane and sulfur and has a refractive index of about 1.78.

However, this immersion oil is insufficient for many applications, since it is relatively volatile at usual operating temperatures, i.e. in particular between about 23° C. and about 37° C., whereby for example longer recording times with the so-called “live cell imaging” technique are rendered impossible. However, in particular the “live cell imaging” technique is of particular interest for the modern cell biology, since hereby microscopic fast motion recordings of moving cells can be made. Moreover, the immersion oil is extremely strong-smelling, no longer transparent below 450 nm and exhibits high intrinsic fluorescence. This property is a disadvantage in particular in microscopic techniques, which assume single molecule detection in the fluorescence. As an example for such microscopic techniques, the so-called “photo activated localization microscopy (PALM)” technique is to be mentioned, which basically allows a resolution down to 20 nm. With the aid of this technique, by localization of single molecules, which can be determined much more exactly than the conventional assessment limitation, an extremely high-resolution image can be successively formed.

### PRESENTATION OF THE INVENTION

It is the object of the present invention to provide an immersion liquid having a refractive index as high as possible with improved suitability for the microscopy at the same time.

This object is solved by an immersion liquid according to claim 1, a method according to claim 12 for preparing an organic compound for an immersion liquid as well as a use of the immersion liquid or of the organic compound according to claim 18. Advantageous configurations with convenient developments are specified in the respective dependent claims, wherein advantageous developments of the immersion liquids are to be considered as advantageous developments of the method and vice versa.

An immersion liquid according to the invention, which has a refractive index as high as possible with improved suitability for the microscopy at the same time, includes at least one organic compound having at least one structural element of the general formula I

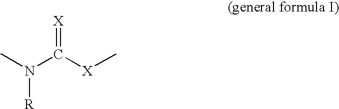

wherein X denotes independently of each other S or O and R denotes hydrogen or hydrocarbon. In other words, the immersion liquid includes at least one organic compound having one or more structural elements from the group of the carbamates, S-carbamothioates, O-carbamothioates and carbamodithioates. Therein, the immersion liquid is in particular suitable for immersion objectives with high NA for microscopes, in particular near-field microscopes, and has a plurality of advantageous characteristics. Thus, the organic compound of the general formula I has a high refractive index n, wherein a simple refractive index adaptation—for example to the employed cover glass material—is allowed due to the variable chemical structure of the compound. Therefore, the optical characteristics of the immersion liquid are adaptable to different operating temperatures, in particular to temperatures between about 23° C. and about 37° C., in optimum manner. Due to the at least one structural element of the general formula I, the immersion liquid is high-boiling and does not or not to an appreciable extent evaporate at the mentioned operating temperatures. Hereby, on the one hand, it is ensured that refractive index variations will not occur during the application in the microscope, on the other hand, the immission values in the operating range are also substantially reduced. Further advantages are in a sufficient wetting of glass surfaces (cover glasses etc.) as well as a high color stability of usual microscopy dyes, since the immersion liquid does not cause decoloration of preparations in operations without cover glass. Thus, the immersion liquid is very well suitable for various microscopic techniques, in particular for the fluorescence microscopy, due to its various advantages. In simplest configuration, the immersion liquid only includes one organic compound, which has at least one structural element of the general formula I. However, basically, it can also be provided that the immersion liquid includes two or more organic compounds each having at least one structural element of the general formula I, thereby providing a particularly simple and precise adaptability of the optical and mechanical characteristics of the immersion liquid to different profiles of requirement.

In an advantageous development of the invention, it is provided that the organic compound has at least two and/or at most five structural elements of the general formula I. Hereby, it is reliably ensured that the organic compound and thus the immersion liquid has a low volatility on the one hand and a sufficient flowability on the other hand in order to be able to be used for microscopic applications.

In further development, it has proven to be advantageous that the immersion liquid has a refractive index n of at least 1.60, preferably of at least 1.70, at 20° C. in the wavelength range between 430 nm and 650 nm. Hereby, the immersion liquid is suitable for immersion objectives with high NA and can be used for microscopic examination of particularly thin layers.

Further advantages arise if the immersion liquid has an Abbe number of at least 18, in particular of at least 19 and preferably of at least 20. In this manner, possible image defects are advantageously minimized or at least largely excluded.

In a further advantageous development of the invention, it is provided that the immersion liquid includes at least one additive, by means of which a viscosity v and/or a refractive index n of the immersion liquid is adjusted to a predetermined parameter value. Hereby, a simple possibility is provided to optimally adapt the viscosity v to the desired operating temperature. Alternatively or additionally, by suitable selection of the type and amount of the additive, a particularly exact and reproducible adjustment of the refractive index to at least Δn±0.001 of the immersion liquid can be performed.

Further advantages arise if the at least one additive includes an alkylnaphthalene, a chloronaphthalene, a bromonaphthalene, an iodonaphthalene, a phenylnaphthalene, a benzylnaphthalene and/or naphthylacetic acid ester. Therein, all of the position isomers of the mentioned additives are to be considered as co-disclosed. By use of one or more of the mentioned additives, the requirements to the immersion liquid regarding dispersion, viscosity, boiling point and wettability of e.g. slides are reliably and inexpensively satisfied. Since the mentioned additives additionally each dispose of at least one ring structure and partially of refractive index increasing functional groups, it is ensured that the immersion liquid also disposes of a high refractive index in case of higher mass fractions of additive. In addition, the mentioned additives are commercially reliably and long-ranging available.

In order to be able to achieve a particularly high refractive index, it has proven to be advantageous in further development if the organic compound having the at least one structural element of the general formula I includes at least one aromatic and/or cyclic, in particular polycyclic, ring structure.

In a further advantageous development of the invention, it is provided that the organic compound having the at least one structural element of the general formula I includes one or more further structural elements from the group of the carboxylic acids, thiocarboxylic acids, carboxylic acid esters, in particular alkyl- and/or arylcarboxylic acid esters, thiocarboxylic acid esters, in particular alkyl- and/or arylthiocarboxylic acid esters, ethers, in particular alkyl- and/or arylethers, thioethers, in particular alkyl-/and or arylthioethers, halides, in particular a chloride, bromide and/or iodide, ketones, thioketones, aldehydes, thioaldehydes, alcohols, thiols, amines and/or amides. This too, presents a simple and flexible possibility of optimally adapt the optical, chemical and mechanical characteristics of the immersion liquid to the respective purpose of employment. Therein, in particular sulfur containing functional groups and halogens (in particular chlorine, bromine and/or iodine) act in refractive index increasing manner.

Further advantages arise if the immersion liquid has a viscosity v of at least 50 mm²/s, in particular of at least 100 mm²/s, in the temperature range between 20° C. and 40° C. In this manner, the immersion liquid has an optimum flowability in particular for microscopic applications.

A further aspect of the invention relates to an immersion liquid including at least one organic compound, wherein the organic compound is obtainable by a reaction between a first compound including at least one isocyanate and/or isothiocyanate group and a second compound from the group of the alcohols and/or thiols. Hereby, the one organic compound has one or more structural elements from the group of the carbamates and/or S-carbamothioates and/or O-carbamothioates and/or carbamodithioates depending on the educts, thereby achieving a refractive index n as high as possible with improved suitability for the microscopy at the same time. Therein, the immersion liquid is in particular suitable for immersion objectives with high NA for microscopes, in particular near-field microscopes, and has a plurality of further advantageous characteristics besides the high refractive index n. Due to the variable chemical structure of the compound, for example, a simple refractive index adaptation—for example to the used cover glass material—is allowed. Hereby, the optical characteristics of the immersion liquid are optimally adaptable to different operating temperatures, for example 23° C. and 37° C. Moreover, the immersion liquid is high-boiling and does not or not to an appreciable extent evaporate at the mentioned operating temperatures. Hereby, it is both ensured that refractive index variations do not occur during the application in the microscope, and that the immission values in the operating range are substantially reduced. Further advantages are in a sufficient wetting of glass surfaces (cover glasses etc.) as well as in a high color stability of usual microscopy dyes, since the immersion liquid does not cause decoloration of preparations in operations without cover glass. Thus, the immersion liquid is very well suitable for various microscopic techniques and in particular for the near infrared microscopy as well as for special applications of the fluorescence microscopy with wavelengths in the range between 380 nm and 1500 nm duo to its various advantages. In simplest development, the organic compound is formed such that it is obtainable by reaction between a monofunctional first and a monofunctional second compound. However, it can also be provided that the organic compound is formed such that it is obtainable by the reaction of a multifunctional first and/or second compound. Moreover, it can be provided that the immersion liquid includes one or more organic compounds obtainable by the reaction between the first and the second compound, thereby providing a particularly simple and precise adaptability of the optical and mechanical characteristics of the immersion liquid to different profiles of requirement.

In an advantageous development of the invention, it is provided that the immersion liquid includes the second compound as a solvent. Hereby, the second compound can be advantageously used as a solvent, whereby the immersion liquid can be prepared in particularly simple and inexpensive manner. By suitable selection of the type and amount of the second compound, the viscosity v and/or the refractive index n of the immersion liquid can be adjusted at the same time. Preferably, the second compound has a boiling point, which is above 100° C., and thus is high-boiling.

A further aspect of the invention relates to a method for preparing at least one organic compound for an immersion liquid, in which the organic compound is prepared by a reaction between a first compound including at least one isocyanate and/or isothiocyanate group and a second compound from the group of the alcohols and/or thiols. Hereby, the one organic compound has one or more structural elements from the group of the carbamates and/or S-carbamothioates and/or O-carbamothioates and/or carbamodithioates depending on the educts, thereby achieving a refractive index n as high as possible with improved suitability for the microscopy at the same time. For avoiding side reactions, it can be provided that the reaction is performed under protective gas atmosphere. Furthermore, it can be provided that the reaction is performed with heating, for example to 50° C.-60° C., to increase the reaction rate. Further arising features and the advantages thereof can be taken from the preceding descriptions.

Therein, it has proven to be advantageous if the reaction includes an addition step between the first and the sec- and compound. Hereby, in simple manner, adducts can be produced from the first and the second compound. Therein, it can further be provided that the reaction additionally includes an addition step between the first compound and a product, which has been formed from the addition of the first and the second compound.

By first providing a predetermined amount of the second compound in a reaction vessel in the preparation and subsequently adding a predetermined amount of the first compound, preferably added dropwise within a predetermined period of time, the reaction can be advantageously directed towards desired products or towards a desired product mixture. In particular in use of a multifunctional first and/or second compound, therein, due to the low local concentration of first compound, the formation of monoadducts from first and second compound is promoted, whereas the formation of multiple adducts is correspondingly repressed. Hereby, in particular, the viscosity v of the immersion liquid can be specifically affected.

In a further development, it can be provided that the second compound is provided in—optionally multiple—molar excess and hereby advantageously serves as a solvent. In addition, hereby, a further simple controllability of the reaction control is provided.

A particularly fast and inexpensive reaction procedure is ensured in further development in that at least one reaction step of the reaction is performed in presence of a catalyst, in particular a tertiary amine.

In a further advantageous development of the invention, a monofunctional isocyanate and/or isothiocyanate is used as the first compound and a compound having at least two and preferably three alcohol and/or thiol groups is used as the second compound for the reaction procedure. Hereby, a product mixture of plural organic compounds can be specifically prepared, wherein the respective organic compounds have one or more structural elements from the group of the carbamates, S-carbamothioates, O-carbamothioates and carbamodithioates depending on the first compound and the number of alcohol and/or thiol groups of the second compound.

A further aspect of the invention relates to a use of one of the immersion liquids according to anyone of the preceding embodiments and/or an organic compound prepared by means of a method according to anyone of the preceding embodiments as an immersion oil for a microscope, in particular for a near-field microscope. The advantages arising from this can be taken from the corresponding descriptions.

Further features of the invention are apparent from the claims and the embodiments. The features and feature combinations mentioned above in the description as well as the features and feature combinations mentioned below in the embodiments are usable not only in the respectively specified combination, but also in other combinations or alone without departing from the scope of the invention.

### PREFERRED IMPLEMENTATION OF THE INVENTION

The following examples illustrate embodiments for organic compounds having at least one structural element of the general formula I and being usable individually or in combination with further compounds as an immersion liquid for microscopes. If required, an additive can additionally be provided for adjusting the viscosity v and/or the refractive index n of the immersion liquid to a predetermined parameter value. Suitable additives for example include alkylnaphthalene, chloronaphthalene, bromonaphthalene, iodonaphthalene, phenylnaphthalene, benzylnaphthalene and/or naphthylacetic acid ester.

Therein, all of the examples satisfy the following requirements:
Refractive index nₑ≧1.70 (546.1 nm, 20° C.)
Abbe number (dispersion) Vₑ≧20
Sufficient transmission in the range between 380 nm and 1500 nm
Low intrinsic fluorescence: F (405 nm/485 nm)<20 mg/l quinine sulfate equivalent
Viscosity at operating temperature>50 mm²/s, in particular >80 mm²/s
Not volatile at usual operating temperatures
Sufficient wetting of glass surfaces (cover glasses etc.)
Color-stable with respect to microscopy dyes (no decoloration of preparations in operations without cover glass)
Long-term availability

By suitable selection and/or combination of the organic compounds as well as optionally of one or more additives, immersion liquids can be provided, which have the following characteristics:
Optical characteristics adjustable to different operating temperatures, in particular between about 23° C. and about 37° C.,
Reproducibility of the refractive index n to n±0.0005
Possibility of refractive index adaptation to the used cover glass material

### EXAMPLE 1

An organic compound (I) for an immersion liquid usable in the microscopy is obtained by reaction between benzylisothiocyanate (n_{D}=1.6036 at 20° C.) as the first compound and 4-mercaptomethyl-3,6-dithia-1,8-octanedithiol (n_{D}=1.6313 at 20° C.) as the second compound according to the general reaction equation

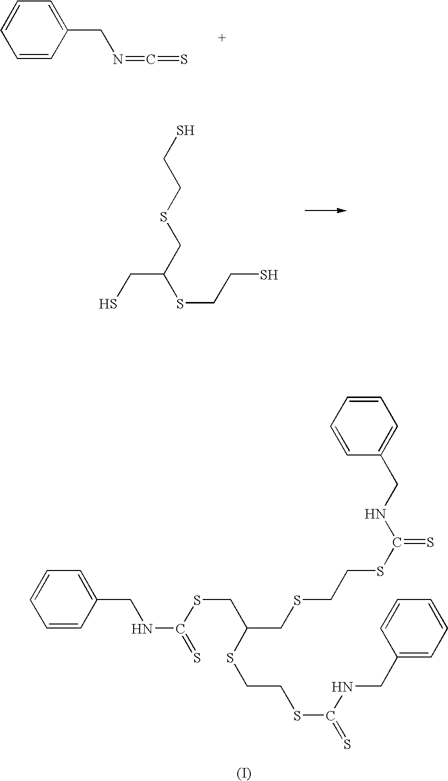

For performing the reaction, first, one mole of the second compound is provided. Subsequently, three moles of the first compound are slowly added dropwise with stirring at a temperature between 50° C. and 60° C. Therein, it can be provided that a catalyst, for example a tertiary amine such as dimethylbenzylamine or hydroxyethylpyrrolidine, is used for accelerating the reaction. Preferably, the reaction is performed under protective gas, wherein for example argon and/or nitrogen can be used as the protective gas. It can also be provided that the second compound is provided in a molar excess and thus functions as a solvent at the same time. Depending on the reaction control, besides the shown organic compound (I), which, as a carbamodithioate, has three structural elements of the general formula I

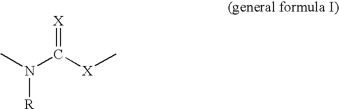

with X=S und R=hydrogen, three mono- and three diadducts also arise in different proportions, which correspondingly have one or two structural elements of the general formula I. In addition, according to the reaction control, low amounts of higher adducts can also occur by reaction between the arisen carbamodithioate groups and benzylisothiocyanate.

Hereby, in particular, the viscosity v and the refractive index n of the immersion liquid can be specifically adjusted. Preferably, the organic compound has at most five and in particular at most three structural elements of the general formula I to ensure a sufficient flowability. Organic compounds with six or more structural elements of the general formula I are normally solid at usual operating temperatures between 20° C. und 40° C. and therefore are normally not suitable or only suitable in small mass fractions as ingredients for immersion liquids.

After completion of the reaction, the organic compound can be purified with the aid of usual methods such as for example column chromatography, preparative HPLC or vacuum distillation, if necessary. The organic compound (I) has the following characteristics:

Refractive indices at 20° C.:
n_{g} (435.8 nm)=1.7430
n_{F′} (480.0 nm)=1.7252
nₑ (546.1 nm)=1.7085
n_{D} (589.3 nm)=1.7008
n_{C′} (643.8 nm)=1.6933

Abbe number (dispersion): Vₑ=22.2

The organic compound (I) as well as the corresponding mono- and diadducts can each be used individually or in mixture as an immersion liquid. For adjusting the viscosity v and/or the refractive index n of the immersion liquid to a predetermined parameter value, in addition, at least one additive can be admixed. Suitable additives for example include alkylnaphthalene, chloronaphthalene, bromonaphththalene, iodonaphthalene, phenylnaphthalene, benzylnaphthalene and/or naphthylacetic acid ester.

### EXAMPLE 2

An organic compound (II) for an immersion liquid usable in the microscopy is obtained by reaction between two moles of phenylisothiocyanate (n_{D}=1.6506 at 20° C.) as the first compound and one mole 4-mercaptomethyl-3,6-dithia-1,8-octanedithiol (n_{D}=1.6313 at 20° C.) as the second compound according to the general reaction equation

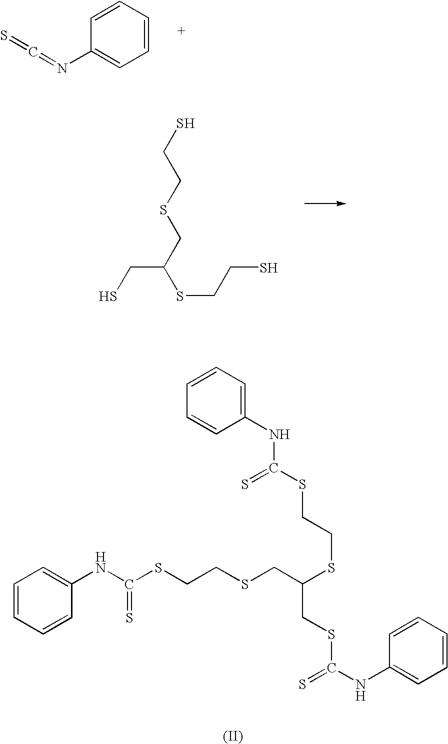

The reaction control is effected in analog manner to the first example. However, due to the relative molar deficit of the second compound, unlike the first embodiment, proportionally more mono- and/or diadducts arise. The organic compound (II) has the following characteristics:

Refractive indices at 20° C.:
n_{g} (435.8 nm)=1.7802
n_{F′} (480.0 nm)=1.7569
nₑ (546.1 nm)=1.7355
n_{D} (589.3 nm)=1.7262
n_{C′} (643.8 nm)=1.7178

Abbe number (dispersion): Vₑ=18.8

### EXAMPLE 3

For preparing an immersion liquid for a microscope, in particular a near-field microscope, 50 vol.-% of organic compound (I) and 50 vol.-% of 1-phenylnaphthalene are mixed. The immersion liquid has the following characteristics:

Refractive indices at 20° C.:
n_{g} (435.8 nm)=1.7251
n_{F′} (480.0 nm)=1.7059
nₑ (546.1 nm)=1.6880
n_{D} (589.3 nm)=1.6801
n_{C′} (643.8 nm)=1.6725

Abbe number (dispersion): Vₑ=20.6

### EXAMPLE 4

Further organic compounds or immersion liquids can be obtained by reaction of any mixtures of the educts listed in table 1. Therein, all of the enantiomers, racemic mixtures and position isomers of the compounds are respectively to be considered as co-disclosed.

TABLE 1

| First compound | Second compound |
|---|---|
| 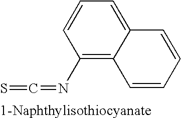 | 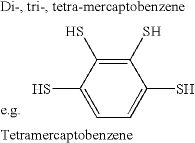 |
| 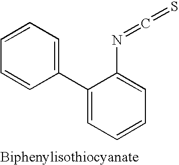 | 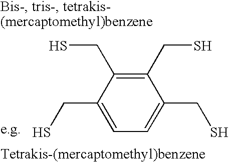 |
| 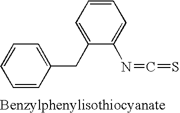 | 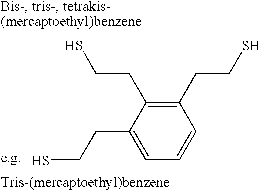 |
| 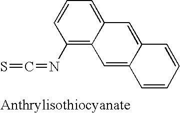 | 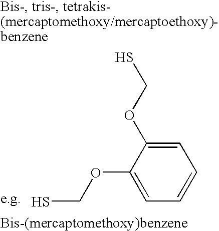 |
| 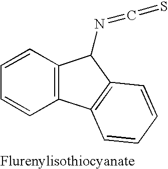 | 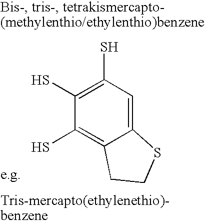 |
| | 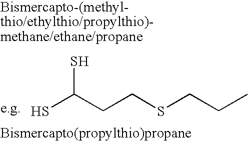 |
| | 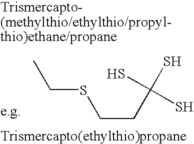 |
| | 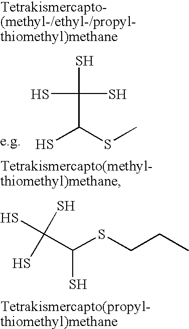 |

Basically, it can be provided that the first compound has an isocyanate group alternatively or additionally to the at least one isothiocyanate group. Furthermore, it can basically be provided that the second compound includes one or more alcohol groups alternatively or additionally to the at least one mercapto group.

Basically, it has proven to be advantageous if the organic compound obtainable from the reaction of the first and the second compound has plural aromatic and/or sulfur containing structural elements in addition to the at least one structural element of the general formula I in order to achieve a particularly high refractive index n.

The parameter values specified in the documents for definition of process and measurement conditions for the characterization of specific characteristics of the subject matter of the invention are to be considered as encompassed by the scope of the invention even within the scope of deviations—for example due to measurements errors, system errors, weighing errors, DIN tolerances and the like.

1. An immersion liquid including at least one organic compound having at least one structural element of the general formula I: 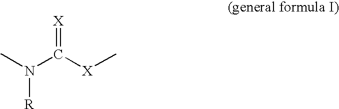
wherein X denotes independently of each other S or O, and R denotes hydrogen or hydrocarbon.

2. The immersion liquid according to claim 1, in which the organic compound has at least two and/or at most five structural elements of the general formula I.

3. The immersion liquid according to claim 1 wherein the immersion liquid has a refractive index n of at least 1.60, at 20° C. in the wavelength range between 430 nm and 650 nm.

4. The immersion liquid according to claim 1, wherein the immersion liquid has an Abbe number of at least 18.

5. The immersion liquid according to claim 1, the immersion liquid further comprising at least one additive, by means of which a viscosity v and/or a refractive index n of the immersion liquid is adjusted to a predetermined parameter value.

6. The immersion liquid according to claim 5, in which the at least one additive includes at least one of the group consisting of alkylnaphthalene, chloronaphthalene, bromonaphthalene, iodonaphthalene, phenylnaphthalene, benzylnaphthalene, naphthylacetic acid ester, and combinations thereof.

7. The immersion liquid according to claim 1, wherein the organic compound having the at least one structural element of the general formula I includes at least one aromatic and/or cyclic ring structure.

8. The immersion liquid according to claim 1, wherein the organic compound having the at least one structural element of the general formula I includes one or more further structural elements selected from the group consisting of carboxylic acids, thiocarboxylic acids, carboxylic acid esters, thiocarboxylic acid esters, thioethers, halides, ketones, thioketones, aldehydes, thioaldehydes, alcohols, thiols, amines amides, and combinations thereof.

9. The immersion liquid according to claim 1, wherein the immersion liquid has a viscosity v of at least 50 mm²/s, in the temperature range between 20° C. and 40° C.

10. An immersion liquid including at least one organic compound obtainable by a reaction between a first compound including at least one isocyanate and/or isothiocyanate group and a second compound selected from the group consisting of alcohols, thiols, and combinations thereof.

11. The immersion liquid according to claim 10, wherein the second compound is a solvent.

12. A method for preparing an organic compound for an immersion liquid, the method comprising reacting a first compound including at least one isocyanate and/or isothiocyanate group and a second compound selected from the group consisting of alcohols, thiols, and combinations thereof.

13. The method according to claim 12, wherein the reaction includes an addition step between the first and the second compound.

14. The method according to claim 12 the method further comprising:
providing a predetermined amount of the second compound in a reaction vessel; and
subsequently adding a predetermined amount of the first compound within a predetermined period of time.

15. The method according to claim 14, wherein the second compound is provided in molar excess with respect to the first compound.

16. The method according to claim 14, in which at least one reaction step of the reaction is performed in presence of a catalyst.

17. The method according to claim 14, wherein the first compound is selected from the group consisting of monofunctional isocyanate and isothiocyanate, and the second compound has at least two alcohol and/or thiol groups.

18. An immersion oil for a microscope comprising the immersion liquid of claim 1.

19. An immersion oil for a microscope, in particular a near-field microscope, comprising an organic compound of claim 10.

20. An immersion oil for a microscope comprising an organic compound prepared by the method of claim 12.

21. The method according to claim 16, wherein the catalyst is a tertiary amine.
